(19) United States
(12) Patent Application Publication
(10) Pub. No.: US 2022/0023374 A1
(43) Pub. Date: Jan. 27, 2022

(54) Integrated Organism Recuperative Treatment Systems and Methods

(71) Applicants: Aleksander Richards, New York, NY (US); Jozef Venglar, New York, NY (US)

(72) Inventors: Aleksander Richards, New York, NY (US); Jozef Venglar, New York, NY (US)

(21) Appl. No.: 17/384,790

(22) Filed: Jul. 25, 2021

Related U.S. Application Data
(60) Provisional application No. 63/056,560, filed on Jul. 25, 2020.

(51) Int. Cl. A61K 36/81; A61K 31/01; A61K 36/53; A61K 36/185; A61K 36/35; A61P 25/20; A61K 36/84

(52) U.S. Cl. CPC A61K 36/81; A61K 31/01; A61K 36/53; A61K 36/84; A61K 36/35; A61P 25/20; A61K 36/185

(57) ABSTRACT

A composition suitable for providing living organisms enhanced rest, recovery and sleep is presented. The composition includes Ashwagandha. The composition also includes LuteMax. The composition further includes Clock. The composition includes Lemon Balm. The composition also includes Lotus Leaf. The composition further includes Valerian Root. A method for producing a composition is also presented. The method adjusts dosages of the ingredients based on an age bracket, gender, stress level a soundness of sleep the person typically has a level of gastric health of the person wherein the dosages chosen by the method include interaction between the person and an online website to determine appropriate dosages.

### FIELD OF THE INVENTION

The present invention relates generally to recuperative systems, and more particularly, to integrated living organism recuperative treatment systems and methods which augment living organism's ability to recuperate in response to natural and environmental conditions.

### BACKGROUND OF THE INVENTION

Many treatments, methods and systems have been used unsuccessfully attempting to provide treatments, systems, or methods to address recuperating living organisms' biological functions because of environmental conditions. These treatments, systems and methods have not been reliable or effective. Many unsuccessful treatments have been offered and are cumbersome and costly.

Throughout the world, the problem of providing adequate remedies to allow proper sleep and rest to all living beings has been a longstanding problem. Quite often local remedies are provided however they do not address all environmental conditions and are not fully effective. In most living beings, the need to sleep and to allow a recovery period is essential. Up to now, the capabilities afforded to provide optimized recuperative periods where living beings can rest and augment their own internal immunology have been limited, and quite often the remedies offered were costly and did not include all the available resources.

In different regions of the world, living beings are exposed to environmental conditions such as electromagnetic pollution, synthetics, processed foods, fossil fuel emissions, and a continuing array of newly introduced energies and materials that affect living beings from being able to recuperate and rejuvenate in response to these factors.

Further, as mankind proceeds to explore areas outside of Earth, the affect different gravitational fields and other electromagnetic forces necessitate the need to have remedies available to replenish living organism's ability to survive. Living beings have innate circadian rhythms that are disrupted and impacted by forces experienced in other parts of the universe. These naturally occurring processes are detrimentally affected by these external factors.

Additionally, as climate change continues to affect planet Earth and continues to create environmental conditions which increase in magnitude and detrimentally affects all living beings, there is a need to provide treatment, systems and methods to address these issues.

Also, most living beings require rest, sleep, hibernation, and/or a state of recuperative condition to survive. As such, there is a need to provide treatment, systems, methods, and processes that can assist in providing living beings with augmented ability to survive in response to the multitude of changing conditions.

These detrimental conditions affect all of society. Because personnel cannot get proper rest, their productivity suffers. These conditions affect all citizens because the cost of these environmental conditions are paid by all the taxpayers because of our society's overall reduced productivity.

In response to an overall societal need for relief for a plurality of physical ills, commercial entities have collectively offered nutritional supplements to address these conditions. The deluge of unregulated commercial offerings claiming relief quite often are inaccurate in their claims and can lead to consumer confusion.

In the past, the pathways that were commonly associated with sleep were oftentimes melatonin production, stress, and the circadian rhythm imbalance. The typical western viewpoint looked at all these pathways in isolation and separate from any interconnectedness. The inability to make further connections with other pathways has limited development of treatment systems and methods that can assist in biological recuperative activity.

In the past, it was thought that only stress and travel could dysregulate the circadian rhythm and that the BMAL1 and SIRT1 genes were the major regulators of the circadian rhythm.

The circadian rhythm is the basis for the production and homeostatic regulation of all hormones relating to sleep such as GABA and melatonin. Commonly used methods to attempt to reset or to restore proper functioning of the circadian rhythm are synthetic substances such as melatonin or GABA or serotonin. Some natural remedies typically revolve around drinking lavender or chamomile tea. However, both common treatments do not get to the root of why the circadian rhythm is dysregulated in the first place whether it be from stress, inflammation, or a compromised gut microbiome.

Accordingly, there is an established need for a living organism treatment system which solves at least one of the aforementioned problems. Further, there is an established need for integrated living organism recuperative treatment systems and methods which can address augmentation of internal biological recuperative functions to allow proper rest and recovery.

### SUMMARY OF THE INVENTION

According to an aspect of the present invention, integrated living organism recuperative treatment systems and methods are provided. These innovations include new and valuable formulations and delivery systems to provide enhanced capabilities for restorative sleep and rest. Innovative and unique formulations are provided to allow human and other beings to allow their internal processes to recuperate, rejuvenate, and recover organic functioning.

According to another aspect of the present invention, processes and methodologies are provided to allow targeted, personalized, and customized recuperative treatment systems and methods to be deployed.

According to yet another aspect, a composition suitable for providing living organisms enhanced rest, recovery and sleep is presented. The composition includes Ashwagandha. The composition also includes LuteMax. The composition further includes Clock. The composition includes Lemon Balm. The composition also includes Lotus Leaf. The composition further includes Valerian Root.

According to still another aspect, a method for producing a composition is presented. The composition comprising dosages of Ashwagandha, LuteMax, Clock, Lemon Balm, Lotus Leaf, Valerian Root, and wherein the method includes determining a person's age and adjusting the dosages based an age bracket of the person. The method also includes ascertaining the person's gender and adjusting the dosages based on the gender of the person. The method further includes calculating a stress level of the person and adjusting the dosages based on the stress level of the person. The method includes finding out a soundness of sleep the person typically has and adjusting the dosages based on the soundness of sleep the person typically experiences. The method also includes measuring a level of gastric health of the person and adjusting the dosages based on the level of gastric health of the person. The method further includes algorithms wherein the dosages chosen by the method include interaction between the person and an online website

In an embodiment of the present invention, compositions of ingredients provided can include Ashwgandha standardized to 10% withanolides 150-500 mg.

In another embodiment, compositions can also include ingredients similar but not limited to LuteMax 2020 in ranges about 200-300 mg.

In yet another embodiment, compositions can include substances similar to but not limited to Clock in ranges of about 500-2000 mg.

In an aspect, compositions can include Lotus Leaf in ranges of about 150-500 mg.

In another aspect, compositions can also include compositions similar to but limited to, Valerian in ranges of about 75-450 mg.

In yet another aspect, compositions can include Lemon Balm in ranges of about 45-350 mg.

In an embodiment, the present invention can provide multi-targeted compositions, formulations, chemical proportions, and combinations to address pathways to regulate sleep and keep living beings in a state of homeostasis.

In another embodiment of the present invention, the system can also include customized algorithms and processes designed to personalize compositions and delivery methods to optimize a person's ability to absorb the individually designed aggregate chemical compounds and to allow a person to biologically recuperate through rest and/or sleep.

In yet another embodiment of the present invention, the system can include applying query routines that are configured to quantify a person's unique biological needs and to calculate appropriate therapeutic options. This will allow for the most precise product recommendation for a person. Each individual has differing levels of stress or other pathway imbalances leading to the fact that not every product will work the same. Using the process to recommend a specific product, we believe, will maximize the therapeutic results.

In an aspect, the system can include online interactive platforms wherein individuals can engage with an algorithm to determine a quantity of at least 5 different boosters configured to balance and improve efficacy of a core product.

In another aspect, the system can also include algorithms based on clinically important timeframes relating to stress, blue light, travel, gut issues, and athletic workouts.

In yet another aspect, the system can include providing an individual the ability to obtain a targeted product configured to boost their body's ability to fight and bring the person's being back into homeostasis and address a dysregulated system. This will be maximized by each ingredient being included at a dosage that has been clinically studied and supported.

In an embodiment of the present invention, the system can include the ability to improve the efficacy of the core product by focusing on once certain area of need while the core product focuses on the holistic approach.

In embodiments, the system can include processes and methodologies which take into account not only individual pathways associated with sleep, but also how these pathways cause other pathways to become unbalanced and contribute to sleep loss. Further, these processes and methodologies transcend previous methods because the present treatment systems are configured with an algorithm which takes into account the interaction between various sleep pathways.

In embodiments, the system can include processes and methodologies to help alleviate creating a cycle of sleep deprivation.

In embodiments, the system can include methodologies and processes which include novel and upstream pathways, these pathways can be crucial regulators and mediators for the promotion of a regulated sleep cycle. Such pathways can include, but are not limited to, inflammation, stress, blue light, and the gut microbiome. These pathways can modulate and effect the circadian rhythm in a non-obvious manner.

In embodiments, the system can include factors in the algorithm which can mitigate stress by providing extra support in the form of a booster product.

In embodiments, the system can include processes which recognize that stress can negatively impact sleep through at least four main pathways. These pathways can include, but are not limited to potential increase in pro-inflammatory cytokines, circadian rhythm imbalance, blue light, which can be a stressor that can increase cortisol, and through increased gap junctions in the epithelial lining of the gut. Each pathway interacts with others in both positive and negative feedback loops and in a synergistically manner. Treating these pathways in a singular effort will not be effective however this is a commonly used approach which is one we will not use.

In embodiments, the system can include algorithms which factor in how individual sleep pathways affect other pathways in becoming unbalanced and contributing to sleep loss.

In embodiments, the system and methods can include algorithms which factors in the affect that stress has on a plurality of pathways, and that each pathway can influence other pathways. Such pathways include, but are not limited to blue light, gut, inflammation, and circadian rhythm.

In embodiments, the system can include processes which provide a higher likelihood of success in restoring biological sleep patterns.

In embodiments, the system can include pathway factors such as but not limited to inflammation. Inflammation is a very new and non-obvious connection that we have identified that can be very important in controlling to achieve an optimal sleep cycle. Inflammation is commonly studied in diseases such as inflammatory bowel syndrome or rheumatoid arthritis but never before with sleep. We have researched and have found that pro-inflammatory cytokines such as TNF-a can be an important biological indicator of insomnia and therefore insomnia can be classified as a type of inflammatory disease. This is a groundbreaking thought process which is very new and novel.

In embodiments, the system can include taking into account TNF-a as a factor. TNF-a levels can also have a positive correlation with many frustrating symptoms of sleep loss such as increased sleep disturbance, decreased sleep duration, and increased daytime dysfunction due to a lack of sleep. A staple of chronic insomnia are increased levels of the proinflammatory TNFa which is caused in part by an activated immune system. Other pro-inflammatory cytokines such as IL-10 also interact with regions of the brain such as the basal forebrain further contributing to a dysregulated sleep cycle. Other pro-inflammatory cytokines such as CRP and IL-6 have also mean implicated in patients who have a short length of sleep, which can be characterized as less than seven hours, which is not only indicative of insomnia but also sleep apneas.

In embodiments, the system can include processes, methodologies which factor in the lowering of CRP. The system can include the CRP mechanism to address treatment protocols. This mechanism is neither well-known nor obvious and we found that higher levels of CRP has the capacity to increase the inability to stay asleep in part due to a hyperactive immune system. Our research shows that for every one-minute increase in sleep onset latency there can be a statistically significant increase in the levels of TNFa. An increased time for sleep onset latency can be a reason why an individual may feel groggy and not fully rested when they wake up. Furthermore, what we found was that people who experienced becoming awake after sleep onset of over sixty minutes had higher levels of the pro-inflammatory IL-6 compared to those who experience wake after sleep onset of less then forty minutes. In individuals who had an anti-inflammatory diet or other forms of intervention it has been shown that their wake after sleep onset had a significant reduction of twenty-five minutes and an improved sleep efficiency score of 2.6%.

In embodiments, the system can include treatments that contain mechanisms which affect the circadian rhythm. The system can include mechanisms that are not commonly known such as the effect of inflammation on the circadian rhythm. The production of pro-inflammatory cytokines and stress hormones such as cortisol are closely connected due to the presence of both positive and negative feedback loops. However, only the cortisol and circadian rhythm connection has been researched. Light cycle desynchronization in addition to a reduction of the expression of a major circadian rhythm regulator in BMAL1 occurs in a situation when inflammatory cytokine secretion is maximized and or increased. In situations where systemic levels of inflammation are increased such as viral infections or other forms of acute infections there is a decrease in the expression of Cry1 which subsequently alters the circadian functioning in a negative manner. Therefore, the notion that the circadian clock is a kind of innate immune sensor whereupon a period of attack whether it be by chronically high levels of cortisol or infections will shift to a pro-inflammatory state can be hypothesized. This shows how the relation of inflammation and the circadian rhythm is a loop where both ends keep the other in check but in periods where the body is stressed the delicate balance can be removed resulting in sleep loss.

In embodiments, the system can include Lotus Leaf in treatment protocols. Lotus Leaf can reduce inflammation much like, but different than Curcumin. Lotus Leaf is less well-known herb that we found still possesses potent anti-inflammatory actions. Lotus has the ability to reduce and modulate both downstream and upstream pro-inflammatory mediators such as IL-6, IL-1b, and TNFa. As has previously been elucidated, downregulating these cytokines has a positive benefit for sleep promotion. Lotus Leaf is also able to downregulate NF-kB signaling which is important as this protects against an overactive immune system. We have found that Lotus Leaf has the capability to influence the production of GABA in a positive manner. We have found that Lotus Leaf has a very powerful effect on sleep promotion predominantly through acting as an anti-inflammatory modulator but also by positively influencing GABA production. This ingredient is an example of how we are thinking outside the box and different from a traditional western medicinal approach. Even though we are using an herb that is part of traditional eastern medicine we are using a non-obvious and much lesser known one when compared to its more commonly well-known adjuncts such as Curcumin.

In embodiments, the system can include factoring in various parameters in customizing treatment protocols. The system can include but is not limited to factoring in connections between the gut, beneficial bacteria and sleep. The connection between the gut, beneficial bacteria, and sleep has been connected before on a very superficial level through a general connection that probiotic supplementation prevents the gut lining from being compromised and therefore allows the absorption of nutrients that when metabolized can exert positive systemic effects. However, there is a much deeper and more important connection to sleep that we have identified that is not obvious. The system can include a non-obvious connection, and that connection is the direct connection that the gut microbiome has with the circadian rhythm and with a potent sleep inducing hormone called GABA. We have identified that the GABA hormone has controls in the gut. This is illustrated in that individuals who have a less diverse and a more germ free microbiome environment have lower levels of GABA. Bacterial species such as Lactobacillus rhamnosus and Bifidobacterium breve have the ability to produce GABA. The system can include non-obvious factors, such as the notion of the gut microbiome affecting the circadian rhythm; this is a very new phenomenon and thinking. In conducting preliminary research of an embodiment of the invention, we saw that over 50% of people who have IBS or Inflammatory Bowel Disease have some form of insomnia or disrupted sleep problems.

In embodiments, the system can include processes, methods and protocols which can take into account the parameter of inflammation. Using the novel notion of inflammation affecting sleep we determined that one mechanism in which the gut microbiome can control the circadian rhythm is through the balance of pathogenic bacteria to beneficial bacteria. We found that pathogenic bacteria are pro-inflammatory and have the ability to over-stimulate the immune system which further compounds the sleep cycle disruption. We proved that microbiome diversity (richness, Shannon diversity, and inverse Simpson diversity) is positively correlated with sleep efficiency, and total sleep time, and was negatively correlated with the sleep fragmentation (WASO). Our research and findings of the present invention contradicts a previously held belief that supported the notion the reciprocal relationship between the microbiome and sleep restriction is insignificantly related to each other. A reason for which our new research on the present invention merits a stronger claim is that we looked at sleep over a time period rather than manipulate sleep by experimentally inducing it. This brings up the concept of the brain-gut-microbiome axis (BGMA).

In embodiments, the system can include processes that take into account several bacterial metabolites that can be identified as possible mechanisms through which probiotic bacteria in the gut are able to communicate with the circadian rhythm. Results of research of the present invention show that a diverse microbiome that includes an ample amount of bacteria from the phyla Bacteroidetes and Firmicutes were positively correlated with sleep efficiency, while only the Bacteroidetes was negatively correlated with sleep fragmentation (WASO). These two bacterial families are also able to modulate the circadian rhythm and genes related to it such as BMAL1. In an embodiment, the system can contain methods which can include identifying the metabolites produced by bacteria that interface through the BGMA. Several human gut associated species in the Bacteroidetes, Actinobacteria, and Firmicutes phyla produce γ-aminobutyric acid (GABA), a neurotransmitter that promotes sleep. Research results of the present invention indicate that the diversity and/or richness of these phyla are generally correlated with healthy sleep. At the taxa level, Corynebacterium also play a role in promoting sleep as they have the ability to potentiate serotonin through the BGMA. Germ-free organisms have significantly different mRNA expression of core circadian clock genes in the liver (Bmal1, Per1, Per2, and Cry1) compared to organisms that have a diverse microbiome.

We have found that mice treated with antibiotics had significantly altered levels of clock genes in the gut and periphery as well as a loss of rhythmicity of gut metabolites compared to wild-type mice. Other factors that are in the gut, which also play a role in microbiome diversity are unconjugated bile acids. These acids have been shown upregulate circadian rhythm genes in an in vitro cellular such as Arnt1, Clock, Npas2, Per1,2,3, Cry1,2.

In embodiments, the system can include Ashwagandha. Ashwagandha is an ingredient that is known in traditional Ayurvedic medicine to have medicinal properties however not all forms of Ashwagandha are efficacious in decreasing markers of stress such as cortisol. Common knowledge will tell most people to use a generic Ashwagandha extract however our research has shown that Ashwagandha standardized to a higher percentage of withanolides will have better efficacy in not only reducing the stress hormone in cortisol but in improving resistance to stress. Both of these mechanisms are important in maintaining a healthy sleep cycle. The traditional medicine community predominantly uses Ashwagandha to assist in sleep due to its cortisol protection properties. However, another less well-known mechanism is that the constituents of Ashwagandha are able to positively act on GABA receptors. We have found that extracts of Ashwagandha have an agonistic effect on GABA receptors in the brain. Ashwagandha also showed higher affinity to GABAρ1 over GABAA receptors. GABAρ1 receptor subtypes are of interest because they are spatially and functionally distinct from other GABAA receptors. Results indicate that Ashwagandha has similar pharmacological efficacy on GABA through a particular constituent called Withanolides. This is a groundbreaking thinking as Withanolides have predominantly been used due to their cortisol reducing properties not for their effects on GABA. It has been supported in numerous studies that Withanolides are the constituent responsible for cortisol reduction. Even though an individual may be getting a full night's sleep that does not necessarily correlate to waking up feeling rested and non-groggy or non-drowsy. We found that an improved outcome for individuals supplemented with Ashwagandha had a self-perceived improvement in alertness after waking up in addition to less drowsiness.

In embodiments, the system can include methods, processes, and protocols which include Blue lights as a factor. Blue light is frequently talked about for eye health however blue light exerts nonvisual effects on the circadian rhythm and ultimately sleep. We have found that recent advances in the understanding of how blue light affects both the circadian rhythm and sleep is through melatonin and a negative phase shift. Previously these pathways have been understood to be connected together however, we learned that these two pathways are separate. The system that controls the suppression of melatonin has a sensitivity that is broadly consistent with the spectral sensitivity of a photopigment called melanopsin. Similarly, the spectral sensitivity of circadian phase shifting shows its maximal effect near the peak spectral sensitivity of melanopsin. However, cones and rods do not play a role in these non-visual effects of light however if there is a contribution it is based on the timing of when there is exposure to light. In general morning light stimulates the clock, while evening and night light delay the clock. However under certain circumstances such as blue light exposure brief flashes of light on the millisecond scale can cause circadian phase shifts. Both melatonin suppression and circadian phase shifts are modulated by the amount of light one is exposed to during the course of a day. “The activity of the circadian pacemaker is aligned to counteract the increasing sleep pressure resulting from sustained wakefulness during daytime. Likewise, the nocturnal increase in circadian sleep tendency counteracts the decrease in sleep propensity resulting from accumulated sleep thereby supporting a consolidated phase of nocturnal sleep”. In the evening i.e. when the circadian system is most sensitive to light-induced phase delays. This means that artificial light such as blue light are able to delay and dysregulate the circadian rhythm clock.

Current treatment options to help with reversing the effects of blue light for sleep include glasses that prevent the absorption of blue light. However in our formulation we have standardized forms of Lutein and Zeaxanthin that haven't been used for sleep before in a product but rather for eye health. It is not obvious to use these ingredients as knowing the relationship between rods and cones absorbing light in the brain and thereby affecting the circadian rhythm requires lots of hard research. The mechanism through which these ingredients are able to promote sleep is not by preventing the absorption of blue light or directly resetting the circadian clock as would be expected but rather it is in reducing oxidative stress and inflammation that in combination and separately dysregulate the circadian rhythm. By counteracting the oxidative and pro-inflammatory effects of blue light the circadian clock can be refined and brought back to homeostasis. These ingredients and the pathways that they target are of the utmost importance considering the amount of time that individuals spend on phones, computers, and other portable devices.

In embodiments of the present invention, treatment protocols include unique and novel formulations customized for each individual. Our formulation multi-targets all the previously mentioned pathways in an attempt to have a long lasting effect rather than a more short term and less efficacious one that results from the supplementation of melatonin or GABA. The resulting mechanism is more like an on off switch. This thinking of targeting not just the circadian rhythm genes but also the effectors of a dysregulated system is not common knowledge. In the research we have found the most common way to bring the circadian rhythm to homeostasis is by identifying ingredients that target BMAL1 or SIRT1. However in taking it a step further, which is a novel thinking, we want to not only modulate genes such as BMAL1 directly but with a multi-targeted approach where we remove any potentially negative triggers such as stress or inflammation that can dysregulate the rhythm. In addition our formula is directly targeting the clock gene with natural ingredients. The circadian rhythm is the basis for the production and homeostatic regulation of all hormones relating to sleep such as GABA and melatonin. Commonly used methods to attempt to reset or to restore proper functioning of the circadian rhythm are synthetic substances such as melatonin or GABA or serotonin. Some natural remedies typically revolve around drinking lavender or chamomile tea. However, both common treatments do not get to the root of why the circadian rhythm is dysregulated in the first place whether it be from stress, inflammation, or a compromised gut microbiome.

In embodiments, the system can include treatments with a Clock ingredient. The Clock ingredient helps to restore circadian rhythm balance by significantly enhancing the expression of Clock genes and factors such as Bmal1 (brain and muscle Arnt-like protein 1), Sirt (Sirtuin 1), F-box and leucine-rich repeat protein 21 (Fbx121) and Fbx13. One of the more important ones is the master circadian gene, BMAL 1. We found that it has been shown to be responsible for detecting and operating on the time of the day signals to help support against pro-inflammatory cytokines. Another novel pathway is the proven notion that oxidative stress can cause potentiate circadian rhythm dysfunction. Other parameters that were positively modulated are BDNF, irisin, acetylcholine (ACH) and melatonin. By naturally stimulating the body's own production of sleep promoting hormones the results will be sustainable long term and exponentially more effective. Acetylcholine is an important marker as it is part of the cholinergic centers of the brain that play a major role in sleep and waking up. The cholinergic system involves the parasympathetic nervous system, which allows Ach to send messages. It is the cholinergic neurons that release Ach. We have found that Ach is important for the brain in an activated state during REM sleep and in wakefulness where one does not feel groggy when waking up. Also related to sleep, Ach is believed to help prevent sleep interruptions, which is regulated by a cholinergic receptor. Ach also operates as a sensor hormone with the ability to filter out biologically important from insignificant stimuli which allows one to have a fuller night's sleep.

In embodiments, the system can include treatments with Valerian. Valerian is an ingredient that has Asian/European roots in holistic herbal medicine being used for sleep effectiveness, however the pathway that we are using it for has a more effect and direct response for sleep effectiveness based on the combination of ingredients in the specified dosages. Valerian's pathway that we are using for the effectiveness of the overall supplement is based on the production of agents that reduce the Rectory Oxygen Species (ROS). Rectory Oxygen Species become present due to the body being exposed to different toxins initiating a inflammatory response. The inflammatory response we found directly affects sleep effectiveness in a negative way. As a result, using the agents Valerian is providing we found we could reduce the inflammation throughout the body through the process aforementioned above. When inflammation is under control the body is not in a greater state of stress than it has to be, therefore achieving homeostatic status.

In embodiments, the system can include treatments and/or formulations with Lemon Balm. Lemon Balm is used in holistic herbal medicine as a relaxer. One of the pathways we are focused on is one in conjunction with the other ingredients specifically with Valerian in specified dosages. Lemon Balm's pathway we have found to be most effective is the production of GABA. GABA is an inhibitory neurotransmitter that reduces the neural excitability within the nervous system. As a result of this reduction the inflammation is reduced in direct correlation. When the inflammation is reduced and given the nature of GABA within the nervous system it was found that cortisol levels are reduced having a positive effect on achieving homeostasis of the circadian rhythm which we found leads to less stress on the body and therefore promoting a state of relaxation. This we achieve relaxation a key component in promoting sleep effectiveness.

In embodiments, the system can include a variation of dosages of ingredients, interacting with each other to achieve optimum rest capability for each individual. For example, Valerian and Lemon Balm have to work in conjunction in a certain ratio of dosages to work effectively with all the other ingredients. In order to achieve this a 2:1 ratio is required for Lemon Balm and Valerian to promote the pathways discussed above. Only then will the pathways work effectively in conjunction to reduce inflammation and promote the homeostatic status of circadian rhythm, both important, and not discussed factors in the herbal significance of these two ingredients.

These and other objects, features, and advantages of the present invention will become more readily apparent from the attached drawings and the detailed description of the preferred embodiments, which follow.

### BRIEF DESCRIPTION OF THE DRAWINGS

The features and advantages of the present invention will be better understood when the Detailed Description of the Preferred Embodiments given below is considered in conjunction with the figures provided.

FIG. 1 is a formulation table for an embodiment of the present invention;

FIG. 2 is an ingredient table and associated target pathways of an embodiment of the present invention;

FIG. 3 is an ingredient and dosage listing for an embodiment of the present invention;

FIG. 4 represents interactions of ingredients, pathways and subsequent effects of an embodiment of the present invention;

FIG. 5 represents methods, processes, and protocols for treatment of an embodiment of the present invention; and

FIG. 6 represents a block diagram showing interactions of ingredients and affects of an embodiment of the present invention.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

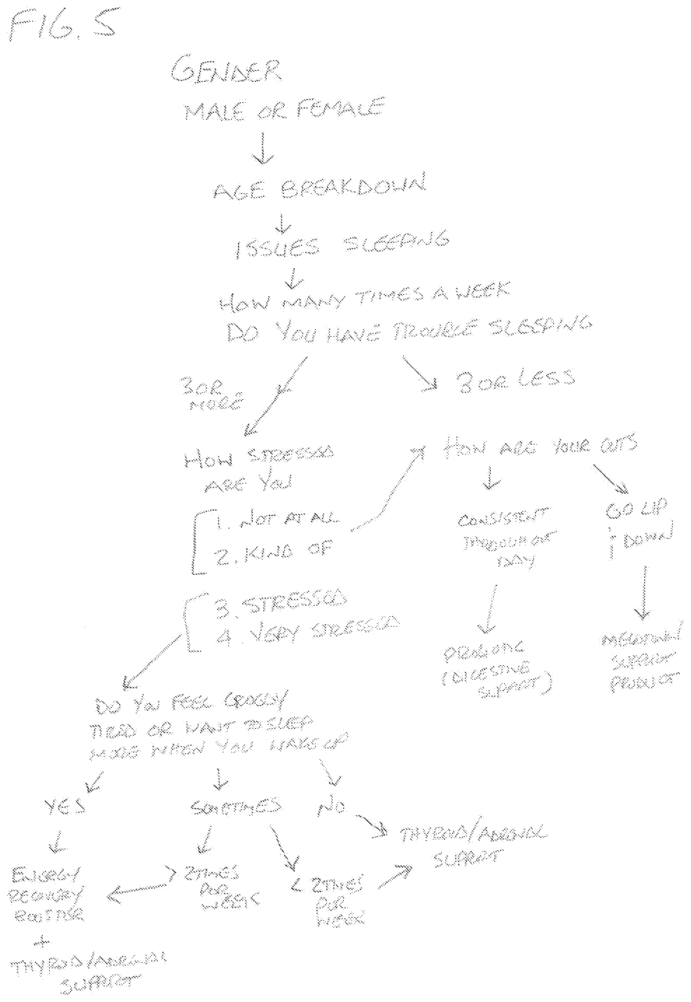

In an embodiment, the system can include methods, procedures and protocols which personalize treatment plans for individuals using answered questions from a user pre-service questionnaire to determine ingredients and dosages. The questions are designed to determine the appropriate dosages from a listing of ingredients associated with specific pathways to allow interaction between the pathways to optimize recuperative benefit. An example of queries presented are shown in FIG. 5.

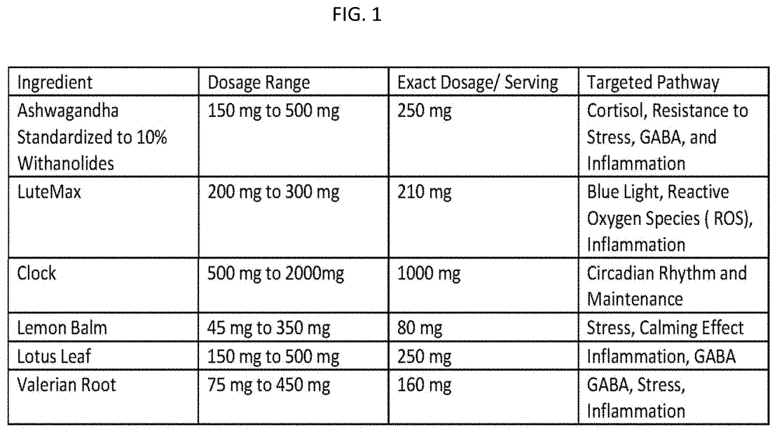

As seen in FIG. 1, a dosage formulation table that is useful for enhancing living organism's recuperative capabilities, in an embodiment, is shown. Ingredients as shown in FIG. 1 have been discovered in an embodiment of the present invention to help people obtain better sleep and rest.

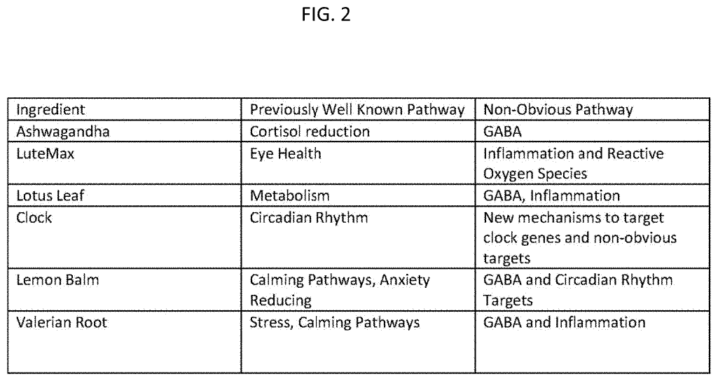

FIG. 2 displays ingredients, pathway interactions and algorithm components of an embodiment of the present invention. The methodologies and protocols shown target specific individuals in obtaining optimum sleep and rest capability.

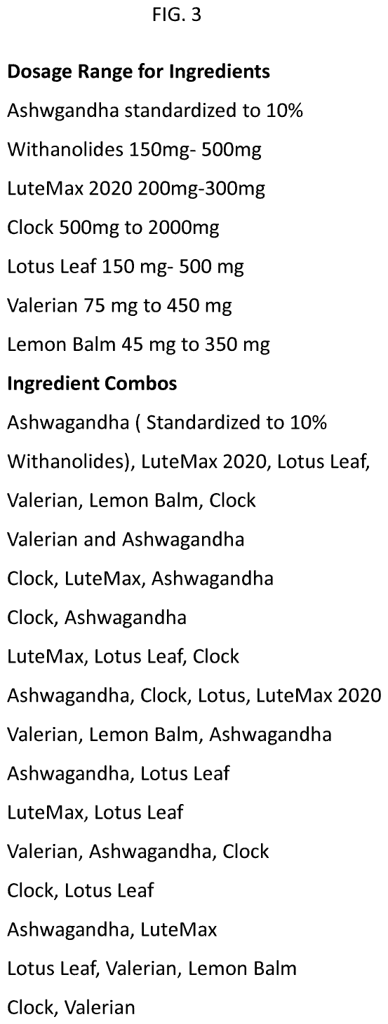

FIG. 3 lists ingredients and compositions which when combined in specific dosages based on methodologies in embodiments of the present invention, assist in augmenting human sleep patterns and rest.

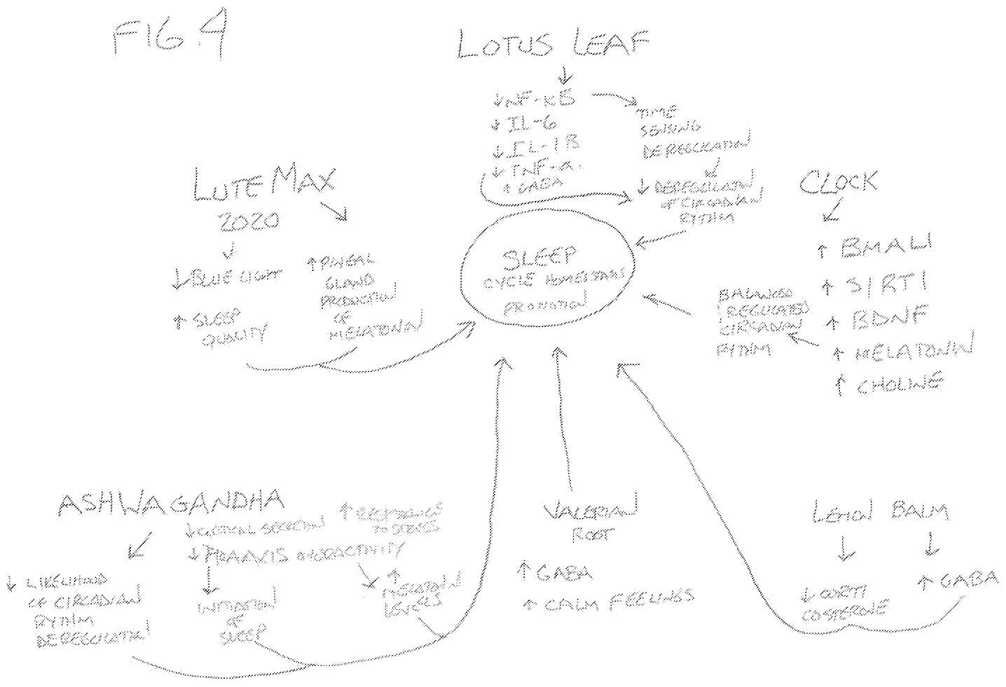

FIG. 4 displays ingredients, pathway interactions and algorithm components of an embodiment of the present invention. The methodologies and protocols shown target specific individuals in obtaining optimum sleep and rest capability.

As best seen in FIG. 5, algorithms of embodiments of the present invention include answering specific questions to queries in order to determine appropriate dosages of a plurality of ingredients. The algorithms are configured to optimize a person's ability to get better sleep and rest.

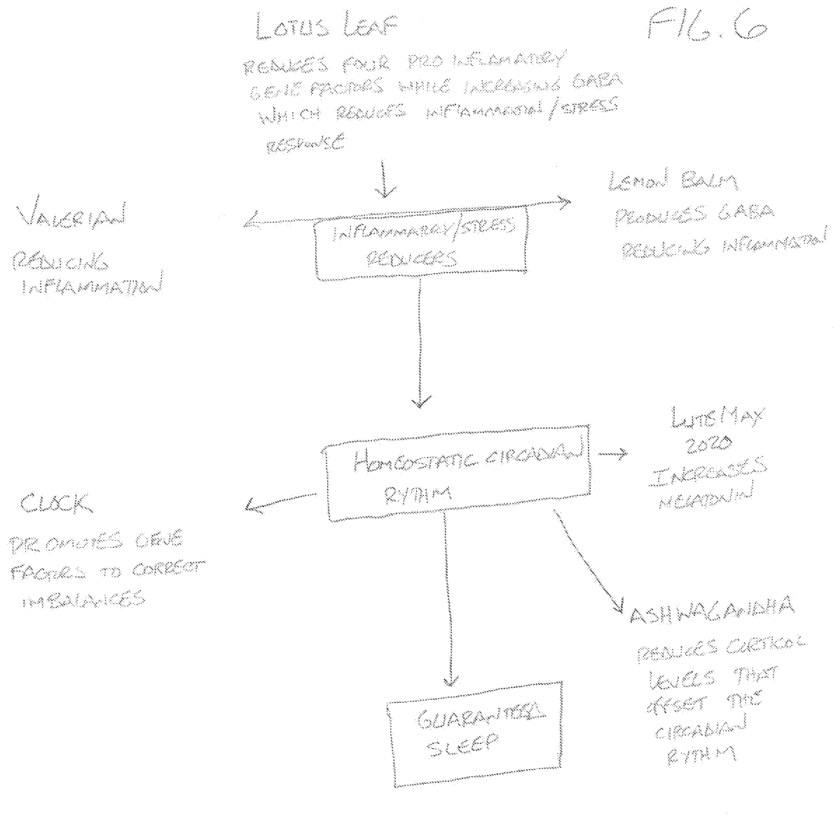

Turning to FIG. 6, ingredients, pathway interactions and algorithm components of an embodiment of the present invention are shown. The methodologies and protocols shown target specific individuals in obtaining optimum sleep and rest capability.

In embodiments, the algorithms can include personalized interaction with an individual to discern factors such as but limited to gender, age, chronic health issues, blood pressure, presence of sleep issues, how tired they feel, how groggy they feel when they wake up, how much stress do they feel.

In embodiments, formulations can include Ingredient Ashwgandha. “Ashwgandha” has a safe dosage range of 150 milligrams (mg) to 500 mg. Depending on the individual's stress levels the appropriate levels of Ashwagandha will be formulated. If an individual is experiencing chronic stress or anxiety their main stress hormone in Cortisol will be significantly elevated. As a result of Ashwagandha being highly effective in lowering levels of stress the higher end of this range will be formulated in their supplement. Higher levels of stress also means that an individual will need to have improved resistance and resilience to stress further supporting the higher dosage range.

In embodiments, formulations can include Ingredient LuteMax. “LuteMax” has a safe dosage range of 200 mg to 300 mg. Depending on the individuals exposure to blue light and their bodies ability to protect itself from the damaging accumulation of free radicals the dosage will be chosen on the higher or the lower end. If an individual has troubles sleeping and spends a lot of time on their phone at night or if an individual has an office job looking at a screen all day the higher end of the range will be prescribed. If an individual is older than the higher end of the range will be prescribed too due to their body's reduced ability to naturally produce antioxidants that can counter the effects of blue light.

In embodiments, formulations can include Ingredient Clock. “Clock” has a safe dosage range of 500 mg to 2000 mg. The dosage will vary depending the amount of time the individual has experienced jet lag and grogginess when they wake up. The more that an individual travels between different time zones the more likely it is that the circadian rhythm pacemakers will be dysregulated and not working in an optimal fashion. The more that a person feels groggy when they wake up means that they are not getting sufficient REM sleep. Supplementing with Clock will rebalance the pacemaker and promote the production of hormones that help you get a full night's sleep.

In embodiments, formulations can include Ingredient Lotus Leaf. “Lotus Leaf” has a safe range of 150 mg to 500 mg. The dosage will vary depending on the self-perceived stress that an individual goes through and the amount of strenuous activity such as running every day that will cause the inflammatory milieu to become pro-inflammatory. The more strenuous activity they do and the more stressed they are the more likely it is that they a high level of inflammation.

In embodiments, formulations can include ingredient Valerian Root. “Valerian Root” has a safe range of 75 mg to 450 mg. The dosage will vary depending on the self-perceived stress that an individual goes through and the amount of strenuous activity such as running every day that will cause the inflammatory milieu to become pro-inflammatory. The more strenuous activity they do and the more stressed they are the more likely it is that they a high level of inflammation.

In embodiments, formulations can include Ingredient Lemon Balm. “Lemon Balm” has a dosage of 45 mg to 350 mg. The dosage will vary depending on the individual's ability to feel restful and be able to feel relaxed at night. There are many factors that one can attribute to not being able to sleep such as higher levels of stress and inflammation (which can cause GABA to decrease) and exposure to lots of blue light. This ingredient will reduce stress and support the healthy production of pro-sleep hormones.

In embodiments not shown, physical attributes of an individual are assessed by human interpretation by a trained medical professional to determine appropriate dosages of ingredients and compositions to be prescribed for an individual. In some embodiments, facial recognition software can be used to assess a person's emotional state and incorporate the person's emotional state data in an algorithm, in order to calculate appropriate ingredient dosages.

In an embodiment of the invention, the system is configured to customize treatment protocols for individuals. The customization can occur on computing devices such as laptops and mobile devices. Also, the system can include mobile applications. The mobile applications configured to customize treatment programs with data in accordance with algorithms derived from interaction with the individual. The mobile applications can be structured to allow system interface amongst all users of the system.

In some embodiments, the systems and/or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, flash drives, cloud storage, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

The present invention has been described with reference to the preferred embodiments, it should be noted and understood that various modifications andvariations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of implementation which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized the scope of this invention is much broader than presented by the claims. It is intended that broader claims will be submitted in an application that claims the benefit of priority from this application.

What is claimed is:

1. A composition suitable for providing living organisms enhanced rest, recovery, and sleep, the composition comprising:
Ashwagandha;
LuteMax;
Clock;
Lemon Balm;
Lotus Leaf; and
Valerian Root.

2. The composition of claim 1 wherein the Ashwagandha is standardized to 10% withanolides.

3. The composition of claim 1 wherein the Ashwagandha includes a dosage range of about 150 mg to about 500 mg.

4. The composition of claim 1 wherein the LuteMax includes a dosage range of about 200 mg to about 300 mg.

5. The composition of claim 1 wherein the Clock includes a dosage range of about 500 mg to about 2000 mg.

6. The composition of claim 1 wherein the Lemon Balm includes a dosage range of about 45 mg to about 350 mg.

7. The composition of claim 1 wherein the Lotus Leaf includes a dosage range of about 150 mg to about 500 mg.

8. The composition of claim 1 wherein the Valerian Root includes a dosage range of about 75 mg to about 450 mg.

9. A method for producing a composition, the composition comprising dosages of:
Ashwagandha;
LuteMax;
Clock;
Lemon Balm;
Lotus Leaf;
Valerian Root;
wherein the method includes
determining a person's age and adjusting the dosages based an age bracket of the person;
ascertaining the person's gender and adjusting the dosages based on the gender of the person;
calculating a stress level of the person and adjusting the dosages based on the stress level of the person;
finding out a soundness of sleep the person typically has and adjusting the dosages based on the soundness of sleep the person typically experiences;
measuring a level of gastric health of the person and adjusting the dosages based on the level of gastric health of the person; and
wherein the dosages chosen by the method include interaction between the person and an online website.
